(19) United States
(12) Patent Application Publication
(10) Pub. No.: US 2011/0106094 A1
(43) Pub. Date: May 5, 2011

(54) MANDIBULAR DISTRACTION SYSTEM AND METHOD OF USE

(76) Inventor: Robert J. Mitchell, Scarborough, ME (US)

(21) Appl. No.: 12/609,668

(22) Filed: Oct. 30, 2009

(51) Int. Cl. A61B 17/60

(52) U.S. Cl. 606/90

(57) ABSTRACT

A mandibular distraction system and method for treating temporomandibular joint disorders by stretching and relaxing the periarticular muscles of the temporomandibular joint. The system includes a head immobilization device, a patient support device and a facial harness. The system also includes upper and lower mouth guards arranged to fit at least partially around the upper and lower teeth. The mouth guards are configured to operate with a chin strap, sliding assembly, and a displacement device. The displacement device may be actuated to apply force to the facial harness by applying pressure on the chin strap, which is converted by the mouth guards to apply a downward uniform pressure on the back of the jaw, stretching and relaxing the periarticular muscles of the temporomandibular joint. The method of the present invention generally provides steps for allowing distraction of the periarticular muscles to occur while the patient is in a passive, supine position with their mouth closed.

### BACKGROUND OF THE INVENTION

1. Field of the Invention

The present invention relates to a mandibular distraction system and a method of using that system to treat temporomandibular joint disorders. More particularly, the present invention relates to a system comprising an adjustable headrest and a facial harness comprising mouth guards and a chin strap which distract the temporomandibular joint and create a stretching and relaxation of the periarticular muscles when pressure is applied to the harness.

2. Description of the Prior Art

The temporomandibular joints (TMJ) serve as hinges for the lower jaw. The TMJ on each side of the cranium is formed between the mandibular fossa of the temporal bone of the skull and the condyle of the mandible. The condyle and fossa are separated by an articular disc which acts as a cushion. Numerous periarticular muscles connect to the mandible, and the masseter, temporal, and medial pterygoid muscles form the mandibular elevators and provide the force necessary to close the lower jaw. The TMJ is susceptible to disorders caused by a tightening of these periarticular muscles and consequent increase in intra-articular pressure. Clenching activates the elevator muscles of the mandible and causes a transient increase in intra-articular pressure. A sustained increase in intra-articular pressure could result from an increase in tonic muscle activity or physical shortening of the periarticular muscles. This pressure can result in extreme discomfort and even eventual degenerative changes from constant compression of the joint surfaces. Patients suffering from TMJ disorders may also suffer from other symptoms as well, such as abnormal joint sounds such as “clicking,” jaw lock, or derangement of the disc and condyle of one or both TMJ joints.

The long-established orthopedic principle of traction, or joint stretching, has been hypothesized to be applicable to the treatment of TMJ disorders caused by tightened periarticular muscles. Specifically, the traction would result in a stretching of the periarticular muscles and a reduction of intra-articular pressure within the TMJ. Increasing the distance between the origin and insertion (the attachment points) of any muscle will increase the length of the muscle beyond its resting muscle tone and can actually increase muscle length. If the length of the periarticular muscles controls the degree of intra-articular pressure, stretching and relaxation of these muscles should not only decrease the severity of relatively minor TMJ disorder symptoms such as clicking, but should also decrease the degree of hard and soft tissue damage caused by increased friction during TMJ engagement. As such, tissue irritation would be decreased and healing facilitated.

To accomplish this stretch a prior attempt suggests the use of flat occlusal splints of such thickness so as to increase the so-called resting length of the mandibular elevators. Pivotal splints have also been recommended for the purpose of placing the TMJ in traction. Both attempts suggest that vertical traction of the joint is theoretically achieved by the placement of a pivot between the joint at the posterior portion of the body of the mandible, the muscles being attached to the more anterior part of the mandible. However, the use of splints or oral appliances may cause certain individuals to clench their teeth more firmly, worsening the condition. Prolonged use of splints or appliances can also lead to changes in the bite or position of the teeth, which can lead to other problems. Furthermore, there have been no controlled studies to confirm the action of these appliances or to determine their effect on TMJ disorders.

Distraction as part of a physical therapy for TMJ disorders has also been suggested. This procedure is usually performed in conjunction with other physical therapy modalities, as well as instructions in a home program aimed at postural correction and muscle relaxation exercises. However, such therapy is extremely uncomfortable for both the caregiver and the patient, as it is generally carried out by hand, and the therapist's thumb(s) are placed as far back as possible on the lower teeth, acting as the fulcrum while the fingers of the same hand pull up on the chin. It is extremely difficult to carry out this procedure for a sufficient time to achieve the desired results, given the uncomfortable positioning of the caregiver and the patient. It is also impracticable for the therapist to apply a consistent pressure adequate to effect TMJ distraction. Furthermore, there have been no randomized controlled clinical outcome studies to determine what effect, if any, this specific procedure has on symptoms of TMJ disorders.

Various devices have been contemplated to effect distraction of the TMJ joint. U.S. Pat. No. 5,746,703 describes a device with two planar members joined by an elastic member which can be opened and closed by the jaw muscles when inserted in the mouth for the intended purpose of relaxing chronically spasmed muscles, which may cause TMJ disorders. U.S. Pat. No. 6,558,392 describes a device for distracting the TMJ using two elongated members which are positioned in the patient's mouth and then forcibly squeezed, driving the teeth apart and distracting the TMJ. Both of these systems require the patient to hold an uncomfortable device in his or her mouth and generally require the patient to awkwardly hold his or her mouth open for periods of time.

As such, it is clear there exists a continuing need in the art for an improved method of treating TMJ disorders by providing a distraction system which successfully and consistently effects a stretching and relaxation of the periarticular muscles of the TMJ. Such a system would increase patient comfort, speed recovery, and substantially improve success rates in relation to other methods of treating TMJ disorders.

### SUMMARY OF THE INVENTION

It is an object of the present invention to provide a distraction system for treating a patient suffering from TMJ disorders. The system specifically enables the caregiver to consistently cause a straight downward traction of the condyle from the fossa, creating a stretching and relaxation of the periarticular muscles.

The present system includes a head immobilization device that includes a patient support and a head rest, the combination of which holds the head at a selectable degree of neck flexion and limits the movement of the head in any direction. The patient support device also allows for the patient's head to be positioned optimally with the long axis of the force to allow for straight downward distraction of the condyle, and parallel with the orientation of the periarticular muscles. A facial harness is removably connected to a sliding assembly and a displacement device. The displacement device includes an actuator, such as a motor, hydraulic cylinder, or the like. The facial harness includes a chin strap which works in conjunction with a pair of mouth guards. The upper and lower mouth guards fit at least partially around the upper and lower teeth, with at least one guard having two or more pivot pins located thereon so that when in position they are near the back of the mouth substantially aligned with or behind the back molars. The pivot pins act together functionally as a fulcrum. The orientation of the mouth guards distributes force along the fulcrum, and an upward force applied to the chin using the harness is converted by the fulcrum and results in downward pressure on the back of the jaw and TMJ, which stretches and relaxes the periarticular muscles.

In one example of the method of the present invention involving the use of the distraction system, a caregiver positions the patient on the support and in the head rest, with the two guards positioned in the mouth and the chin strap placed around' the patient's chin. The displacement device is then actuated to cause the chin strap to move upwardly towards the patient's upper jaw. This causes the patient's mouth to close upon the guards. With the mouth fully closed and the constant and uniform pressure applied to the patient's chin, the pivot pins of the guard cause the back of the mandible to move in a relative downward direction, resulting in a stretching of the periarticular muscles.

It is another object of the present invention to provide a method for treating TMJ disorders by distracting the TMJ of an afflicted patient using the distraction system described above. The method may be carried out for the purpose of treating a patient who is suffering from a TMJ disorder resulting from overly tight periarticular muscles. Use of this method enables more effective stretching of the muscles in comparison to known methods of TMJ distraction. The system and method of the current invention are configured so as to substantially improve success rates and increase patient comfort when used as described herein for the treatment of TMJ disorders.

These and other objects of the present invention will be more fully understood upon review of the following detailed description, the accompanying drawings, and the appended claims.

### BRIEF DESCRIPTION OF THE DRAWINGS

FIG. 1 is a front view of the mandibular distraction system of the present invention, showing the adjustable head immobilization device, the chin strap, and a three point harness attached to the sliding assembly.

FIG. 2 is a side view of the mandibular distraction system of the present invention, showing the sliding assembly and the displacement device.

FIG. 3 is a rear view of the mandibular distraction system of the present invention.

FIG. 4 is a side view of the system in use in a method of relieving a TMJ disorder in a patient.

FIG. 5A is a bottom view of a first type of an upper mouth guard of the present invention. FIG. 5B is perspective view of a first type of a lower mouth guard of the present invention

FIG. 5C is a top view of a second type of upper mouth guard of the present invention. FIG. 5D is a perspective view of a second type of lower mouth guard of the present invention.

FIG. 6A is a side view of the guard of FIG. 5A, and FIG. 6B is a side view of FIG. 5B.

FIG. 6C is a side view of the guard of FIG. 5C, and FIG. 6D is a side view of FIG. 5D.

### DETAILED DESCRIPTION OF THE PREFERRED EMBODIMENTS OF THE INVENTION

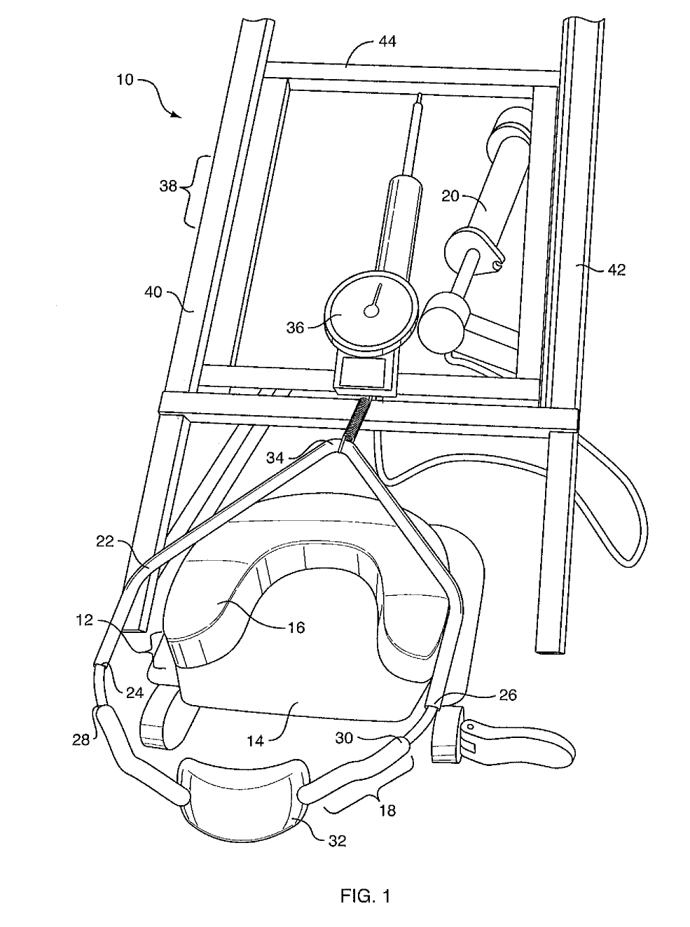

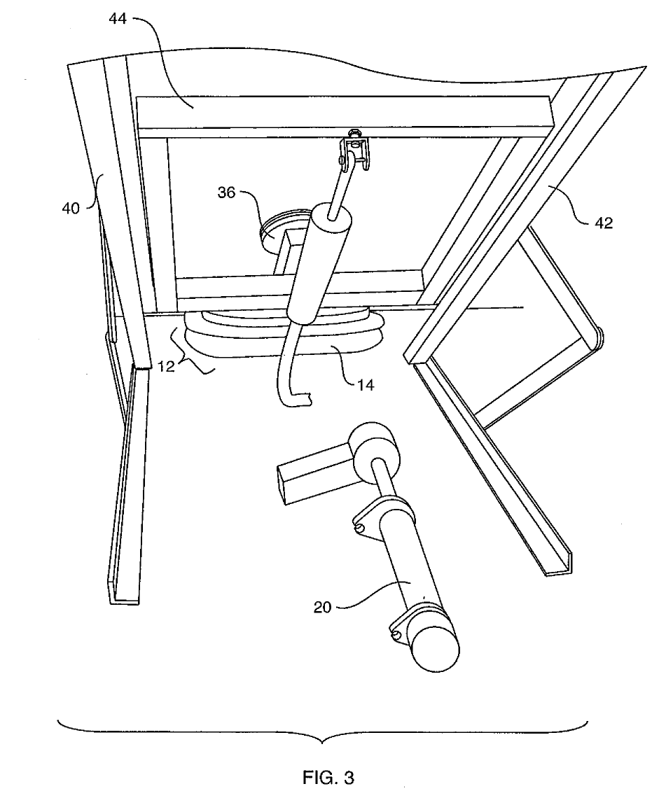

Referring to FIGS. 1-4, a mandibular distraction system 10 of the present invention includes an adjustable head immobilization device 12 which is attached to a platform 14 made of any material suitable for use as a patient support. The platform 14 and head immobilization device 12 are configured so as to keep a patient in a supine position while holding the patient's head at a slight degree of neck flexion most suitable for TMJ distraction as determined by the caregiver. In one embodiment, the head immobilization device 12 includes a head rest 16 joined with the platform 14 configured to hold the head at a desired angle.

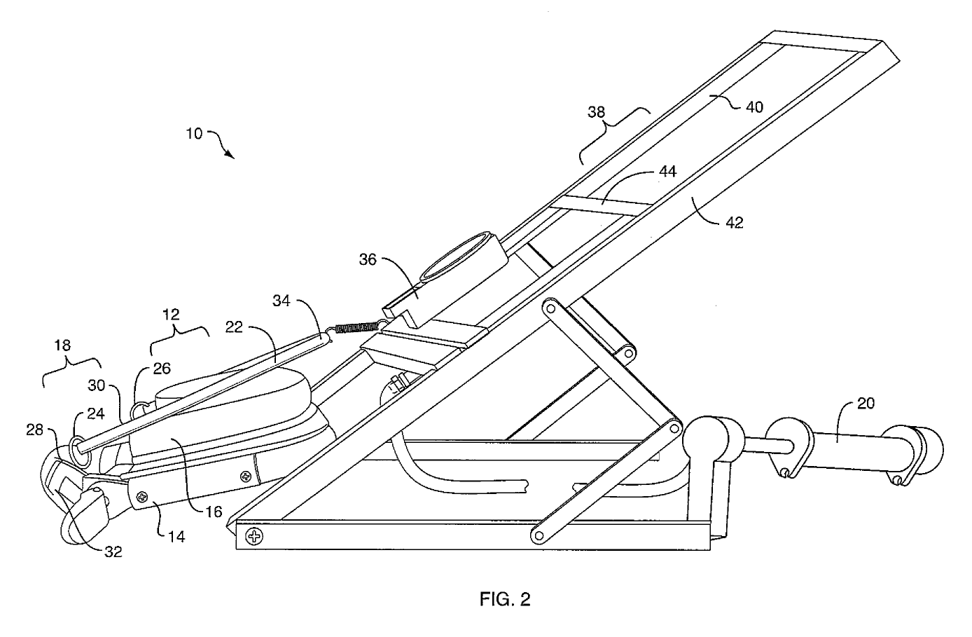

The distraction system 10 includes a facial harness 18 configured to apply upward pressure to the chin of the patient when used in conjunction with a displacement device 20. In one embodiment, the facial harness 18 includes a three-point frame 22 (see FIGS. 1 and 2) wherein two points 24, 26 of the frame are removably connected to opposing ends 28, 30 of the chin strap 32, and the third point 34 is removably connected directly, or indirectly through an optional pressure gauge 36, to a sliding assembly 38 and displacement device 20. Any controllable actuator may operate as the displacement device 20, including a motor, hydraulic cylinder, or the like. It may also be operated manually. The displacement device 20 may be attached to the platform 14 of the distraction system or may be separate from the platform 14.

The sliding assembly 38 is removably connected to the facial harness 18. The sliding assembly 38 includes two tracks 40, 42 wherein a center piece or bar 44 is arranged for slidable movement on the tracks 40, 42 based on force applied by the displacement device 20, which is coupled to the sliding assembly 38. In one embodiment, the sliding assembly 38 is attached to the head rest 16 and the platform 14 at substantially the same angle as the patient's head and extends back behind the head (see FIG. 2). The angle of the sliding assembly 38 may be fixed or adjustable. Any suitable materials or design may be employed for use as the sliding assembly 38. A similar arrangement may be made of hard plastic, such as plastic tubing with a sliding frame attached to a collar. A different configuration could be the use of an A-frame using a block and tackle arrangement to pull upwards on the chin harness.

Activation of the displacement device 20 causes the center piece 44 attached to the facial harness 18 to slide back and away from the patient, applying upward pressure on the patient's chin through the chin strap 32 of the facial harness 18. Applying upward pressure allows jaw distraction to occur while the patient's mouth is closed, providing a substantial improvement in effective uniform distraction over the methods and systems of distraction known in the prior art as described. This improvement in patient comfort also allows the patient to use the distraction system 10 for longer periods than other methods known in the art, allowing for more rapid progress in distraction and subsequently more rapid relief of symptoms of TMJ disorders.

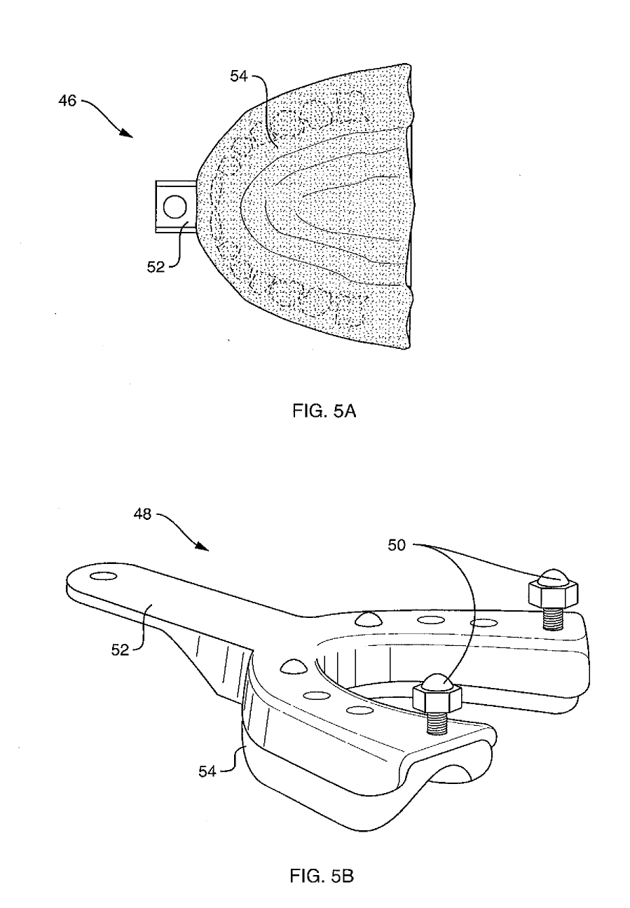

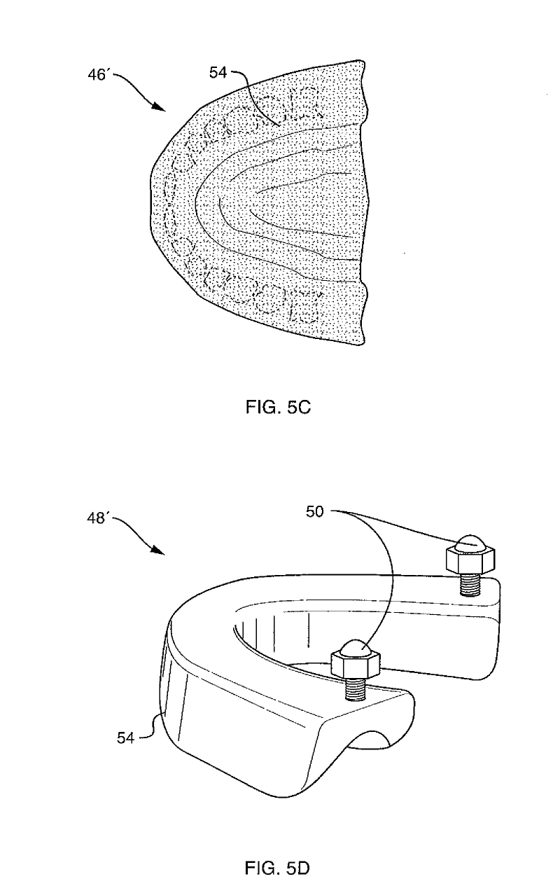

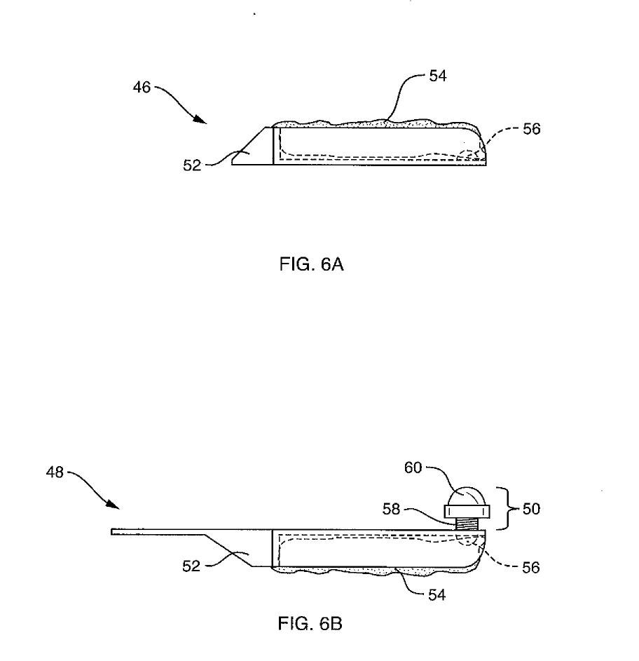

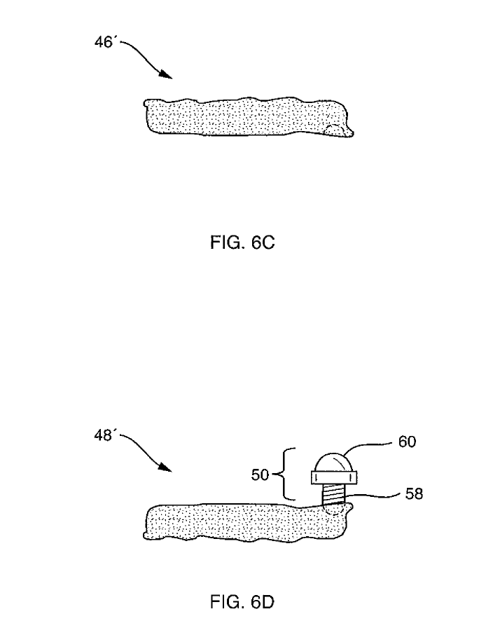

The distraction system 10 also includes a pair of mouth guards, one upper guard 46 and one lower guard 48, that fits at least partially around the upper and lower teeth (see FIGS. 5A-D, 6A-D). Each guard 46, 48 includes a forward section and a rearward section, with at least one guard 46 or 48 having two or more pivot pins 50 in the rearward section. When the guards 46, 48 are in position in a patient's mouth, the pivot pins 50 are located near the back of the mouth and are substantially aligned with or behind the back molars. The pivot pins 50 act as a fulcrum. In one embodiment, the distraction system 10 includes only a single mouth guard, such as the lower mouth guard 48 and the lower mouth guard 48 includes a forward section and a rearward section with two or more pivot pins 50 in the rearward section. The mouth guards 46, 48 are made of any suitable material known in the art. In a first embodiment the guards 46, 48 may include a stock disposable plastic impression tray 52 which is filled with a boilable thermoplastic material or a stable impression material 54 such as a polysilicone which then stabilizes the tray 52 on the upper teeth as shown in FIGS. 5A, 5B, 6A, and 6B. In a second embodiment the guards 46′, 48′ may be any appliance which fits over the upper teeth and has a hard occlusal surface with which to engage the lower pivot 50, such as a standard acrylic “stabilization splint” or night guard as shown in FIGS. 5C, 5D, 6C, and 6D. The function of the upper guard 46 is to resist and distribute the force of point contact with the lower guard 48. The lower guard 48 is composed of a stock impression tray 52 which includes a threaded hole 56 in the most posterior occlusal area of the tray 52 which receives a nylon bolt 58 which is screwed in from within the inside of the tray 52 and the head of which is secured to the inside (impression side) of the tray 52. The bolt 58 acts as the pivot pin 50. The outer end of the nylon bolt 58 protrudes from the occlusal surface of the tray 52 and is capped with a nylon “acorn” nut 60 which is adjustable along the limits of the protruding bolt 58. The lower tray 52 of the lower guard 48 can then be custom fitted as with the upper tray 52 of the upper guard 46.

The orientation of the mouth guard or guards 46, 48 when in position in the mouth distributes force along the fulcrum, and the upward force applied to the chin is converted by the fulcrum and causes downward pressure on the back of the condyle, which stretches and relaxes the periarticular muscles of the TMJ. The relaxation of the periarticular muscles resulting from the use of the distraction system 10 is a passive process for the patient, and a clear improvement over devices known in the art.

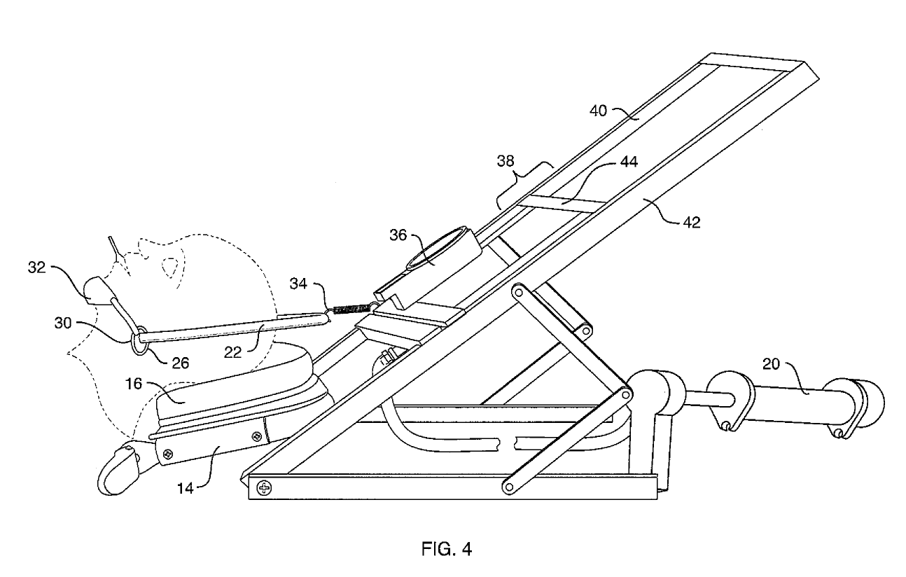

The method of the present invention may be carried out for the purpose of treating a patient who is suffering from a TMJ disorder. The method generally includes steps for allowing for TMJ distraction to occur while the patient is in a passive, supine position with their mouth closed, as shown in FIG. 4. Specifically, the method includes a first step of a caregiver positioning the patient on the platform 14 and the head immobilization device 12 so that the patient is in the most appropriate position for TMJ distraction as determined by one skilled in the art. The method also includes as a second step the caregiver positioning one or both of the mouth guards 46, 48 in the patient's mouth and placing the facial harness 18 on the patient. The method also includes as a third step the caregiver activating the displacement device 20 attached to the facial harness 18 so as to apply uniform upward pressure to the patient's chin and cause movement of the patient's jaw toward their head, causing the patient's mouth to close upon the guards 46, 48. With the mouth fully closed and constant and uniform pressure applied to the patient's chin, the pivot pins 50 of the guards 46, 48 cause the back of the mandible to move in a relative downward direction, resulting in a stretching of the periarticular muscles of the TMJ. The pressure gauge 36 may be used to fix the pressure applied to the chin by the facial harness 18. The pressure may be maintained for a selectable period of time.

The method of treating TMJ disorders using the distraction system 10 of the invention can be employed alone, or in conjunction with additional treatment steps such as the use of nighttime splints, ultrasound, alterations in diet, the use of moist heat, elimination of harmful habits, and posture correction.

The present invention has been described with respect to various examples. Nevertheless, it is to be understood that various modifications may be made without departing from the spirit and scope of the invention. All equivalents are deemed to fall within the scope of this description of the invention.

What is claimed is:

1. A system for distracting the temporomandibular joint of a patient, comprising:
a head immobilization device;
a facial harness;
upper and lower mouth guards arranged to fit at least partially around the upper and lower teeth, wherein on of the mouth guards includes a fulcrum-establishing component;
a sliding assembly attached to the head immobilization device and the facial harness; and
a displacement device removably connected to the sliding assembly for applying force to the facial harness to cause the mouth to close on the upper and lower mouth guards when placed on the upper and lower teeth of the patient.

2. The system of claim 1, wherein the facial harness is a three-point frame.

3. The system of claim 2, wherein the facial harness includes a chin strap and wherein two points of the three-point frame are removably connected to opposing ends of the chin strap, and the third point is removably connected to the sliding assembly.

4. The system of claim 1, wherein the facial harness is directly connected to the displacement device.

5. The system of claim 1, wherein the facial harness is indirectly connected to the displacement device via a pressure gauge.

6. The system of claim 1, wherein the one of the mouth guards includes two or more pivot pins as the fulcrum-establishing component.

7. The system of claim 6, wherein the pivot pins are substantially aligned with or behind back molars of a patient when the mouth guards are placed in the patient's mouth to establish a fulcrum around which the jaw pivots.

8. The system of claim 6, wherein the mouth guards are oriented so as to distribute force along the fulcrum and the upward force applied to the chin is converted by the fulcrum and causes a downward pressure on the back of the patient's jaw and the temporomandibular joint, which stretches and relaxes the periarticular muscles.

9. The system of claim 1, wherein the angle of the sliding assembly is fixed or adjustable.

10. The system of claim 1, wherein the sliding assembly includes two tracks and a center piece attached to the displacement device, wherein the center piece is configured to slide on the tracks.

11. A system for distracting the temporomandibular joint of a patient, comprising:
a head immobilization device and a patient support device;
a facial harness;
a mouth guard arranged to fit at least partially around the lower teeth and including a fulcrum-establishing component;
a sliding assembly attached to the head immobilization device and the facial harness; and
a displacement device removably connected to the sliding assembly for applying force to the facial harness to cause the mouth to close on the mouth guard when placed on the lower teeth of the patient.

12. The system of claim 11, wherein the facial harness is a three-point frame.

13. The system of claim 12, wherein the facial harness includes a chin strap and two points of the three-point frame are removably connected to opposing ends of the chin strap, and the third point is removably connected to the sliding assembly.

14. The system of claim 11, wherein the mouth guard includes two or more pivot pins, as the fulcrum-establishing component.

15. The system of claim 16, wherein the pivot pins are substantially aligned with or behind back molars of a patient when the mouth guard is placed in the patient's mouth to establish a fulcrum around which the jaw pivots.

16. The system of claim 14, wherein the mouth guard is oriented so as to distribute force along the fulcrum and the upward force applied to the chin is converted by the fulcrum and causes a downward pressure on the back of the patient's jaw and the temporomandibular joint, which stretches and relaxes the periarticular muscles.

17. A method for treating TMJ disorders using a distraction system including a head immobilization device and patient support device, a facial harness, an upper mouth guard, a lower mouth guard, a chin strap, a sliding assembly, and a displacement device for applying force to the facial harness by applying pressure on the chin strap, the method comprising the steps of:
positioning a patient on the platform and head immobilization device;
positioning the mouth guards in the patient's mouth and placing the facial harness on the patient; and
activating the displacement device attached to the facial harness so as to apply upward pressure to the patient's chin, causing the patient's mouth to close upon the guards and causing the back of the mandible to move in a relative downward direction, resulting in a stretching of the periarticular muscles of the TMJ.

18. The method of claim 17, wherein the facial harness includes a three-point frame.

19. The method of claim 18, wherein two points of the three-point frame are removably connected to opposing ends of the chin strap, and the third point is removably connected to the sliding assembly.

20. The method of claim 17, wherein the facial harness is directly or indirectly attached to a pressure gauge.

21. The method of claim 17, wherein at least one of the mouth guards includes two or more pivot pins which act as a fulcrum.

22. The method of claim 21, wherein the pivot pins are substantially aligned with or behind the back molars when the mouth guards are placed in the mouth.

23. The method of claim 21, wherein the mouth guards are oriented so as to distribute force along the fulcrum and the upward force applied to the chin is converted by the fulcrum and causes a downward press on the back of the jaw and the temporomandibular joint, which stretches and relaxes the periarticular muscles.

24. The method of claim 17, wherein the angle of the sliding assembly is fixed or adjustable.

25. The method of claim 17, wherein the method is used in conjunction with one or more treatments selected from the group consisting of: the use of nighttime splints, ultrasound, alterations in diet, the use of moist heat, elimination of harmful habits, and posture correction.

26. A method for treating TMJ disorders, the method comprising the steps of:
establishing a fulcrum at the back of a patient's mouth about which the jaw pivots;
maintaining the patient's mouth in a closed position;
applying pressure to the patient's mouth while closed; and
causing the back of the mandible to move in a relative downward direction, resulting in a stretching of the periarticular muscles of the TMJ.
